(19) United States
(12) Patent Application Publication
(10) Pub. No.: US 2004/0152641 A1
(43) Pub. Date: Aug. 5, 2004

(54) Use of polymethoxylated flavones for treating insulin resistance

(76) Inventors: Najla Guthrie, London (CA); Elzbieta Maria Kurowska, London (CA)

Correspondence Address: Santosh K. Chari, McCarthy Tetrault LLP, 66 Wellington Street West, Suite 4700, P.O. Box 48, Toronto, ON, M5K 1E6, CA

(21) Appl. No.: 10/697,563

(22) Filed: Oct. 31, 2003

Related U.S. Application Data
(63) Continuation of application No. PCT/CA02/00662, filed on May 2, 2002.
(60) Provisional application No. 60/287,703, filed on May 2, 2001.

(51) Int. Cl. A61K 31/7048; A61K 31/353

(52) U.S. Cl. 514/27; 514/456

(57) ABSTRACT

Compositions and methods for treating metabolic abnormalities arising from insulin resistance comprises the administration of trangeretin or a mixture of various polymethoxylated flavones (PMF's) are described. The PMF's are administered in various manners including orally. Supplementation with PMF's to individuals affected by insulin resistance syndrome results in normalization of metabolic activity and improved glucose metabolism.

This application is a Continuation of PCT Application No. PCT/CA02/00662 filed on May 2, 2002, which claims priority from U.S. Provisional Application No. 60/287,703 filed on May 2, 2001.

### BACKGROUND OF THE INVENTION

1. Field of the Invention

The present invention relates to the use of polymethoxylated flavones (PMFs) for treating the effects of insulin resistance syndrome.

2. Description of the Prior Art

Insulin resistance is defined as an impaired ability of insulin to stimulate glucose uptake and lipolysis and to modulate liver and muscle lipid metabolism. In animals and humans, insulin resistance syndrome leads to compensatory hyperinsulinemia and to various defects in lipid metabolism such as enhanced secretion of atherogenic, triacylglycerol-rich very low-density lipoproteins (VLDL), increased liberation of nonesterified fatty acids (NEFA) from adipose tissue and increased accumulation of triacylglycerols in the liver¹. Other metabolic defects associated with insulin resistance include impairment of endothelium-dependent vasodilation. This last abnormality is largely a consequence of reduced bioavailability of nitric oxide, an important biological mediator involved in protection against atherosclerosis².

Insulin resistance syndrome commonly precedes type 2 diabetes and both disorders are associated with increased risk of heart disease. Dietary strategies designed to diminish this risk are currently not well established. The most common approach is the recommendation to lower intake of total calories, especially fat and sugar, and to increase intake of fiber³.

The present inventors have recently shown that polymethoxylated flavones, or polymethoxyflavones, (PMFs) from citrus fruits, especially tangeretin (5,6,7,8,4′-pentamethoxyflavone) from tangerines, have hypolipidemic potential in cells and in animals. Flavonoids are polyphenolic compounds that are found in plant foods, especially in oranges, grapefruits and tangerines. PMFs are flavonoid compounds having multiple methoxy substituents. Various beneficial effects of flavonoids are described in U.S. Pat. Nos. 6,251,400 and 6,239,114 and in PCT publication number WO/01/70029, issued to the present inventors and the disclosures of which are incorporated herein by reference. Other beneficial effects of flavonoid derivatives are discussed in U.S. Pat. Nos. 4,591,600; 5,855,892; and, 6,096,364, the disclosures of which are also incorporated herein by reference.

The present inventors have shown that in human liver cell line HepG2, tangeretin substantially reduced production of apolipoprotein B (apo B), the structural protein of VLDL and LDL. This was associated with inhibition of synthesis of cellular lipids, especially triacylglycerols and cholesteryl esters, and with decreased cellular accumulation of triacylglycerols. The apo B-lowering effect of tangeretin was also maintained in the presence of excess of oleic acid, a NEFA known to stimulate cellular biosynthesis of neutral lipids for assembly and secretion of apo B-containing lipoproteins in the liver⁴. These results suggested that tangeretin affected lipoprotein metabolism through multiple mechanisms. In animal studies using hamsters with casein-induced hypercholesterolemia, 0.13-1.0% supplementation with tangeretin significantly reduced serum content of triacylglycerols and cholesterol, however, this was not associated with reduced accumulation of liver triacylglycerols⁵.

There exists a need to provide a safe and effective method of treating the deleterious effects of insulin resistance.

### SUMMARY OF THE INVENTION

The present invention provides, in one aspect, a method of treating hyperlipidemia comprising the use of a polymethoxyflavone.

In another aspect, the invention provides a use of a polymethoxyflavone as a hypolipidemic agent.

More specifically, the invention provides for tangeretin as the above mentioned polymethoxyflavone.

### BRIEF DESCRIPTION OF THE DRAWINGS

These and other features of the preferred embodiments of the invention will become more apparent in the following detailed description in which reference is made to the appended drawings wherein:

FIG. 1 illustrates the effect of tangeretin on apo-B responses in in vitro studies.

FIG. 2 illustrates the effect of tangeretin on serum total cholesterol in hamsters.

FIG. 3 illustrates the effect of tangeretin on HDL cholesterol responses in hamsters.

FIG. 4 illustrates the effect of tangeretin on serum trigylceride responses in hamsters.

FIG. 5 illustrates the effect of tangeretin on serum NEFA responses in hamsters.

FIG. 6 illustrates the effect of tangeretin on serum insulin responses in hamsters.

FIG. 7 illustrates the effect of tangeretin on serum nitrate/nitrite levels in hamsters.

FIG. 10 illustrates a general structure of flavonoid compounds.

FIG. 11 illustrates the effect of PMFs on alpha-glucosidase activity in vitro.

FIG. 12 illustrates the effect of experimental diets on serum cholesterol levels.

FIG. 13 illustrates the effect of experimental diets on serum triacylglycerol and NEFA levels.

FIG. 14 illustrates the correlation between serum triacyglycerol and NEFA levels.

FIG. 15 illustrates the effect of PMFs on glucose tolerance.

### DESCRIPTION OF THE PREFERRED EMBODIMENTS

The present invention provides compositions and methods for treating metabolic defects associated with insulin resistance, otherwise referred to as insulin resistance syndrome, in mammals and, more particularly, humans. The compositions of the present invention comprise PMFs that are obtained from natural sources, and, therefore, are readily available and are generally non-toxic when administered in acceptable dosages as described below.

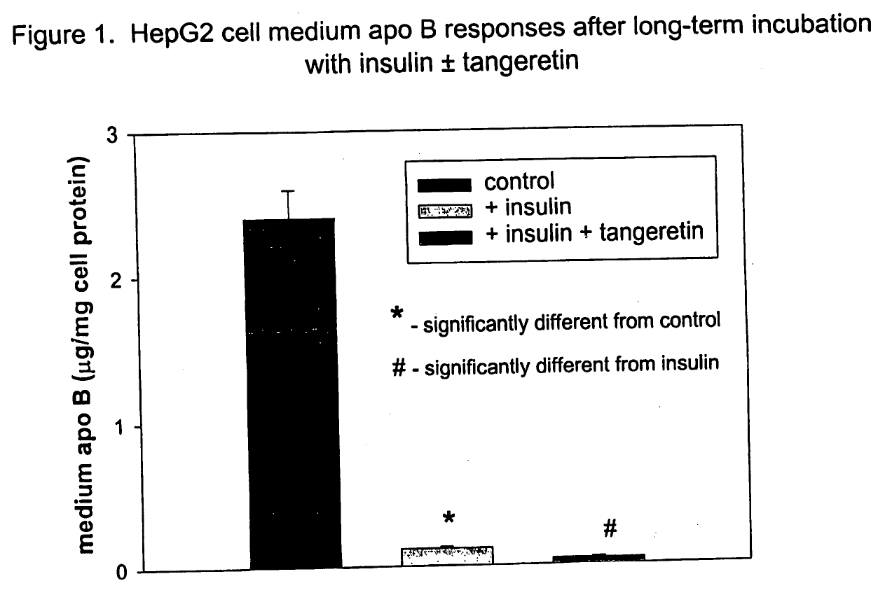

FIG. 1 illustrates a general structure for the flavonoids of the present invention. The following table identifies various flavonoid compounds based on the respective substituents:

| Compound | R5 | R6 | R7 | R8 | R2′ | R3′ | R4′ | R5′ |
|---|---|---|---|---|---|---|---|---|
| Tangeretin | OCH₃ | OCH₃ | OCH₃ | OCH₃ | H | H | OCH₃ | H |
| Nobiletin | OCH₃ | OCH₃ | OCH₃ | OCH₃ | OCH₃ | H | OCH₃ | H |
| Hesperetin | OH | H | OH | H | H | OH | OCH₃ | H |
| Naringenin | OH | H | OH | H | H | H | OH | H |

As a general definition, a polymethoxylated flavones, or polymethoxyflavone (PMF), are flavones substituted with two or more methoxy groups. PMFs can include two to seven methoxy groups. Optionally, PMF compounds are also substituted with one or more hydroxy groups. As can be seen in the above table, tangeretin and nobiletin fall withing the above PMF definition. Hesperetin and naringenin are members of the group of flavonoids referred to as flavonones.

The amount of the PMFs of the administered to a patient will depend on various factors. Acceptable dosages of the PMFs of the invention may be up to 5000 mg/day. Preferable dosages range from 200-5000 mg/day, commonly 1000-2000 mg/day, and typically 500-1500 mg/day. On a patient basis, the dosage of the PMFs maybe up to 70 mg/kg/day, based on the weight of the patient. Patient dosages may range from 15-70 mg/kg/day, commonly 15-30 mg/kg/day and typically 7-21 mg/kg/day. As will be understood by persons skilled in the art, the dosage administered to the patient will depend on a number of factors such as the severity of the condition being treated, the age and weight of the patient etc. As such, the above mentioned dosage ranges should be considered as a guideline and should not be construed as limiting the scope of the invention.

Formulations containing the PMFs of the present invention may by administered by any acceptable means including orally, transdermally, rectally, intravenously, intramuscularly, intraperitoneally, subcutaneously, topically, by inhalation or any other means. The oral administration means is preferred. Formulations suitable for oral administration are commonly known and include liquid solutions of the active PMF compounds dissolved in a diluent such as, for example, saline, water, PEG 400 etc. Solid forms of the compounds for oral administration include capsules or tablets, each comprising the active ingredients and commonly known adjuvants. The active ingredients in the solid dosage form may be present in the form of solids, granules, gelatins, suspensions, and/or emulsions, as will be apparent to persons skilled in the art.

Formulations suitable for parenteral administration include aqueous and non-aqueous isotonic sterile solutions containing buffers, antioxidants, preservatives and any other known adjuvants.

As will be understood, the PMFs of the invention can be administered as a single dose or in a sustained release formulation.

In one embodiment, the present invention comprises the use of a mixture of PMFs as the therapeutically effective active ingredient. In another embodiment, the invention comprises the use of tangeretin as the sole active ingredient.

The following examples serve to illustrate the present invention and are not meant to be construed as limiting the scope of the invention in any way.

### EXAMPLE 1

### Effect of Tangeretin in Treating Insulin Resistance Syndrome

As discussed above, it has been shown that tangeretin reduced pathological responses known to be associated not only with hypercholesterolemia but also with insulin resistance (hypertriglyceridemia, high plasma free fatty acids and possibly high triacylglycerols in liver cells). For this reason, its effect was investigated in cell culture and animal models of insulin resistance. In cell culture studies, the hypolipidemic potential of tangeretin was evaluated using HepG2 cells made insulin-resistant by long-term incubation with high concentrations of insulin⁶. In vivo, metabolic responses to increasing doses of tangeretin were determined using hamsters made insulin resistant by feeding 60% fructose diet⁷.

a) In Vitro Studies

In the cell culture study, 80-90% confluent HepG2 cells were incubated for 5 days with the following media:

1. Minimum essential medium containing 1% bovine serum albumin (MEM+BSA)

2. The same medium containing 1.0 mM bovine insulin

3. The same medium containing 1.0 mM insulin and 25 μg/mL of tangeretin

All media were changed on day 3 to maintain high concentration of insulin (which undergoes partial degradation after long-term incubation). After 5 days, media and cells were collected. Medium concentrations of apo B were measured by Elisa and expressed as μg per mg cell protein as described previously⁸.

The results (as illustrated in FIG. 1) demonstrate that a long-term incubation of HepG2 cells with high concentration of insulin reduced medium apo B by 95%, in accordance with previous reports⁶. In cells exposed to both insulin and tangeretin medium apo B was reduced further (by 69% when compared to insulin alone). The results suggested that tangeretin might be effective as hypolipidemic agent in the insulin-resistant state.

b) In Vivo Studies

In the animal study, hamsters (8-10 animals each) were given semipurified, 60% fructose diet with or without 0.25%, 0.5% or 1.0% tangeretin, and the control group was fed a standard semipurified diet which did not produce insulin resistance. Diets were pair-fed to control for 2 weeks. After that time, fasting blood samples were collected by heart puncture for measurement of plasma lipids, glucose, NEFA, insulin and nitrites/nitrates (end products of nitric oxide metabolism). Total cholesterol in whole serum and in HDL fraction as well as total triglycerides and glucose were measured by enzymatic timed-endpoint methods, using the Beckman Coulter reagents and SYNCHRON™ LX System. VLDL+LDL cholesterol concentrations were calculated as a difference between total and HDL cholesterol. NEFA were determined enzymatically by NEFA C kit (Wako Chemicals USA Inc., Richmond, Va.). Serum insulin was measured using Rat Insulin RIA kit from Linco Research Inc. St. Charles, Mo. Serum nitrates/nitrites concentrations were determined using Nitrate/Nitrite Colorimetric Assay kit from Cayman Chemical Co., Ann Arbor, Mich.

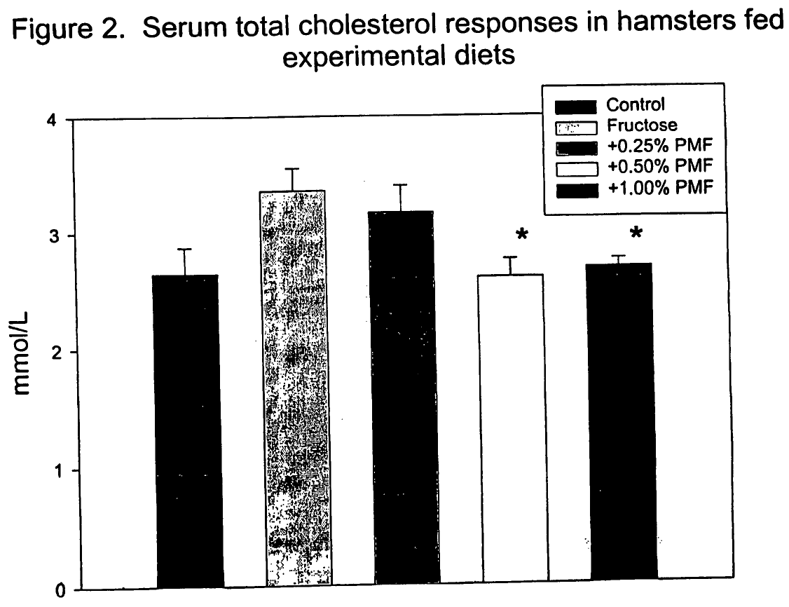

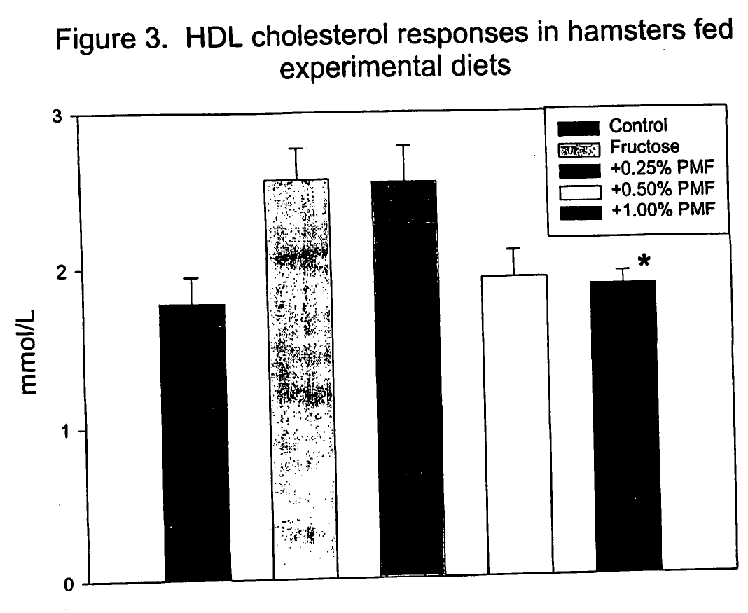

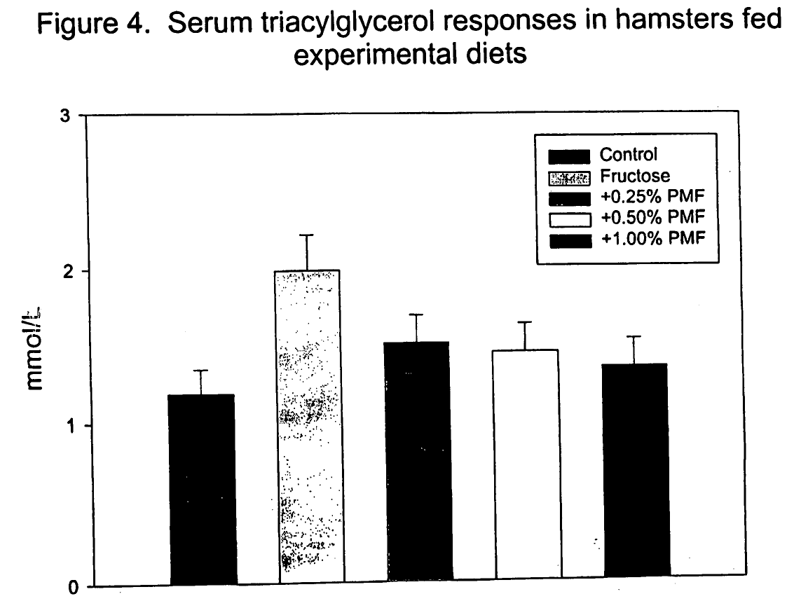

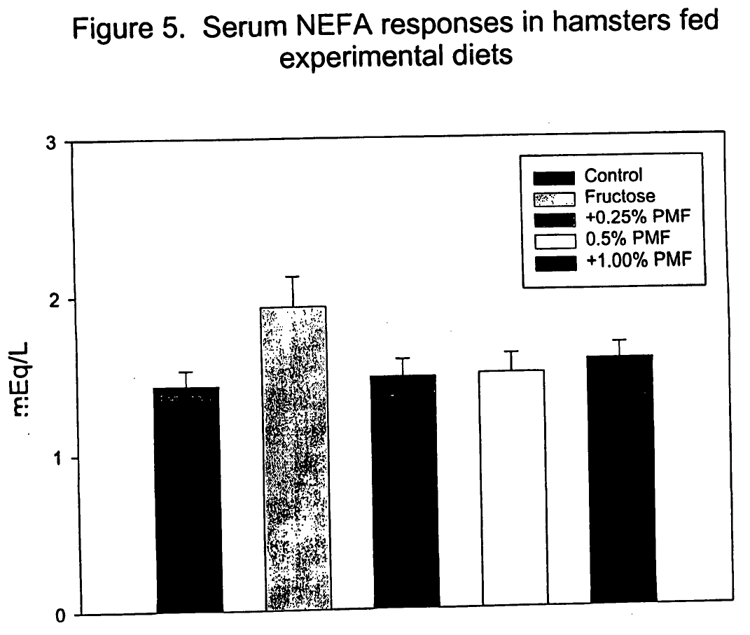

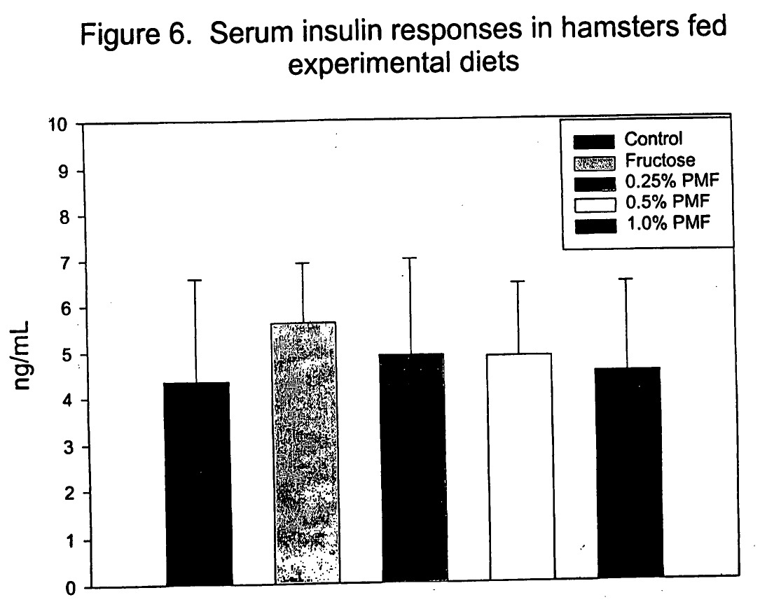

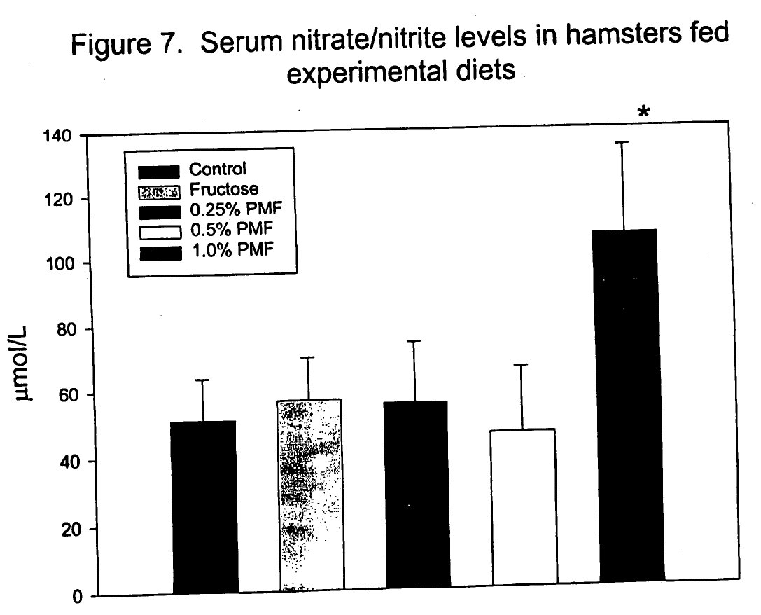

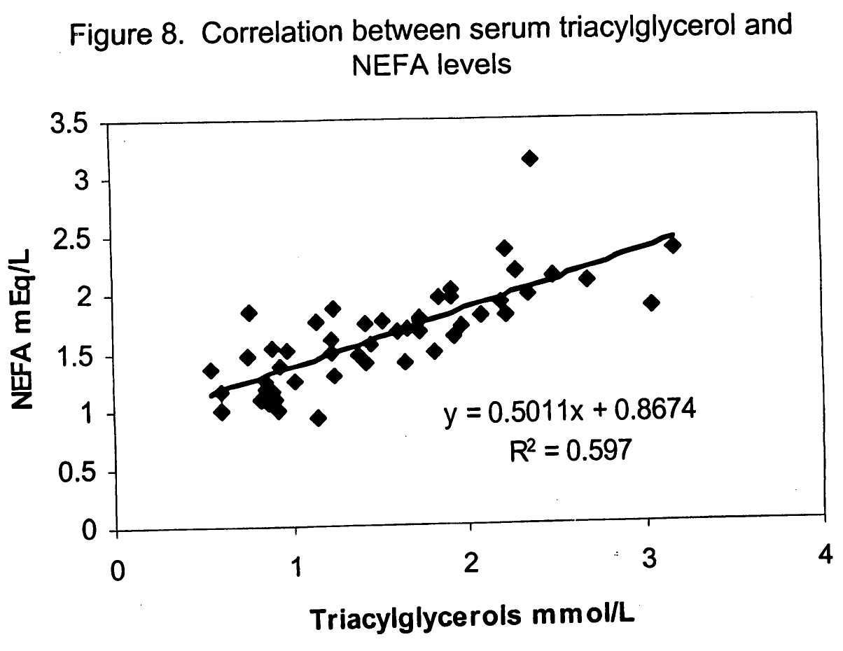

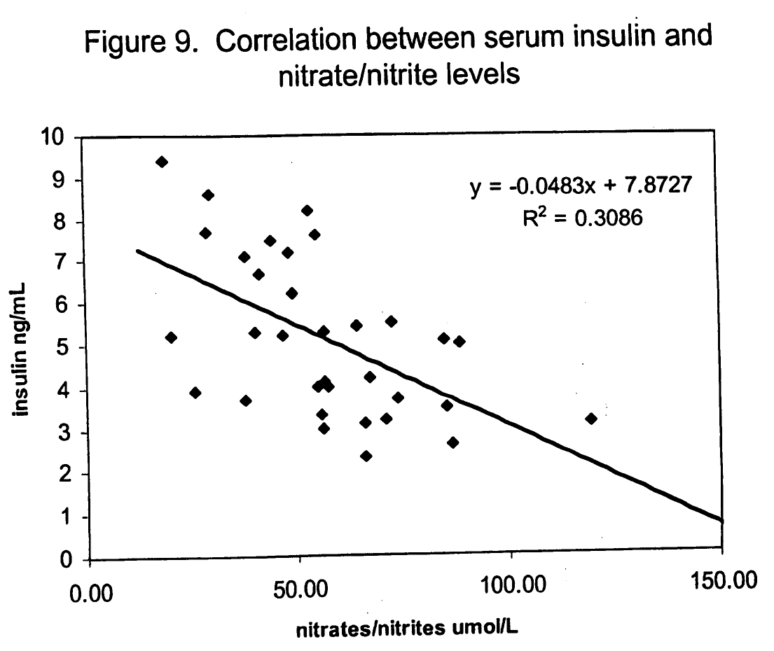

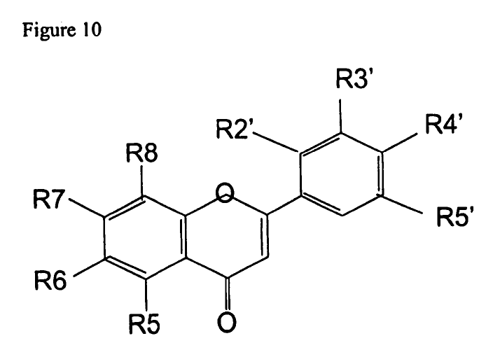

As indicated in Table 1, the growth performance data showed no significant difference in growth rate and food consumption between the groups. Replacing control diet with 60% fructose resulted in moderate increases in serum total and HDL cholesterol, triacylglycerols, NEFA and insulin (by 26%, 44%, 67%, 35% and 29%, respectively). These increases were either partly or completely reversed by supplementation with tangeretin as indicated in Table 2 and FIGS. 2 to 7. The fructose-induced increases in serum total cholesterol were reversed by 0.5% and 1.0% tangeretin, the increases in HDL cholesterol were reversed by 1.0% tangeretin and the increases in serum total triacylglycerols tended to be reversed by all three levels of tangeretin as illustrated in FIGS. 2 to 4. In addition, at all three levels of supplementation, tangeretin tended to normalize serum NEFA concentrations. A diet containing 1.0% tangeretin also tended to normalize serum content of insulin. Serum nitrate/nitrite concentrations were not affected by fructose feeding but their concentration was doubled in the group given fructose with 1% tangeretin. Serum glucose was not altered by fructose feeding or by supplementation with tangeretin. As illustrated in FIGS. 8 and 9, in all dietary groups, serum NEFA concentrations were highly positively correlated with serum triacylglycerol levels (r²=0.597) but not with other parameters measured. Serum insulin levels were inversely correlated with nitrate/nitrite (r²=−0.309).

The results of the animal study demonstrate that hamsters fed 60% fructose diet developed metabolic abnormalities consistent with insulin resistance and that these abnormalities were partly or completely abolished by 0.25-1.0% supplementation with tangeretin. The dietary fructose-induced increases in serum total cholesterol, triacylglycerols, NEFA and insulin were less pronounced than those reported earlier^{1, 6}. This was likely because in our study, unlike in the earlier ones, animals were pair-fed to prevent excessive weight gain in groups given fructose. The cholesterol- and triglyceride-lowering effects produced by tangeretin supplements were similar to those observed in our earlier studies using hamsters with experimental hypercholesterolemia. However, in the insulin-resistance model, tangeretin additionally tended to normalize elevated serum levels of NEFA and insulin. The beneficial effect of tangeretin on serum NEFA could be associated with its ability to modulate triacylglycerol metabolism, as suggested by the significant positive correlation between serum NEFA and serum triacylglycerol levels. In contrast, a tangeretin-induced tendency to normalize serum insulin could be linked to its ability to raise the systemic level of endothelium-derived nitric oxide. Indeed, recent studies in rats with fructose-induced insulin resistance and in patients with type 2 diabetes postulated a functional coupling between insulin resistance and endothelial nitric oxide production^{9, 10}. Also, in our experiment, the inverse correlation was found between serum levels of insulin and nitric oxide metabolites.

### EXAMPLE 2

### Effect of a Mixture of PMFs in Treating Insulin Resistance Syndrome

The following studies were conducted to investigate the efficacy of a mixture of PMFs in treating insulin resistance syndrome.

a) In Vitro Studies

Additional in vitro studies were conducted to determine whether tangeretin, other polymethoxylated flavones (PMF) as well as common flavanones and mixed coumarins found in citrus might help to achieve normal blood glucose levels in patients with insulin resistance and diabetes type 2 by inhibiting activity of alpha-glucosidase, the enzyme that catalyzes the final step in the digestive process of carbohydrates. Previous studies showed inhibition of this enzyme by other natural flavonoids including apigenin and luteolin but excluding hesperidin, a glucoside of citrus flavanone hesperetin¹¹.

For the assay, alpha-glucosidase Type 1 from bakers yeast was incubated for 30 min, at 37° C., in the presence of substrate (p-nitrophenyl-alpha-D-glucopyranoside) and in the presence vs. absence of citrus flavonoids or coumarins at concentrations ranging from 3 to 200 μg/mL (0.01 to 1.8 mM). The reaction was stopped by addition of 0.2 M Na₂CO₃ and absorbance was measured at 405 nm. Background absorbance (without enzyme) was subtracted for every flavonoid or coumarins concentration used. The inhibitory activity was expressed as percent control and IC₅₀ values (concentrations of compounds required to inhibit alpha-glucosidase by 50%) were calculated.

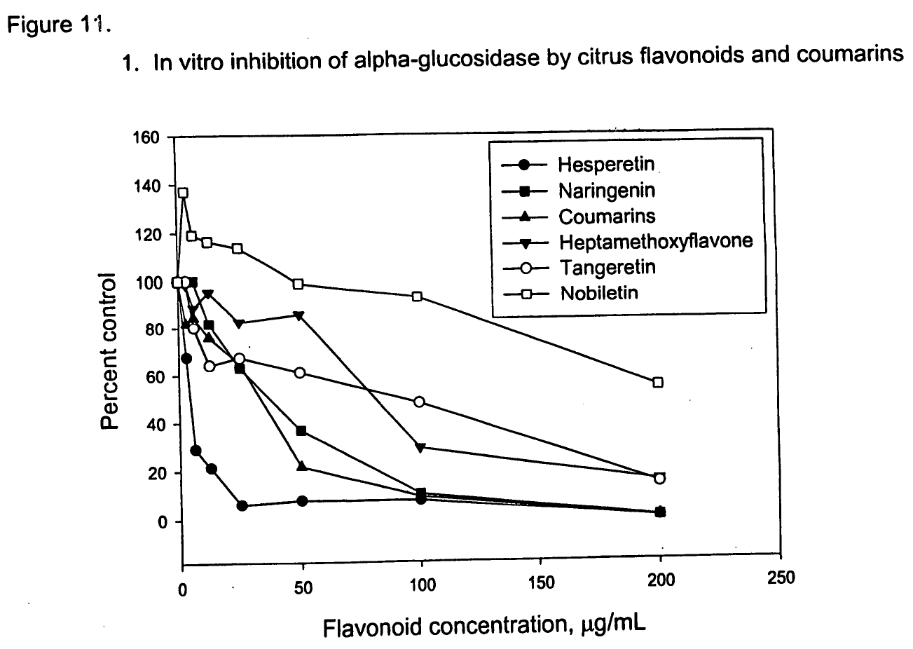

As illustrated in FIG. 11 the results show that all citrus PMF, flavanones and coumarins produced a dose-dependent inhibition of alpha-glucosidase. According to IC₅₀ values presented in Table 3, hesperetin, coumarins and naringenin were the most active, heptamethoxyflavone and tangeretin produced intermediate inhibitory effects and the activity of nobiletin was the lowest. The most pronounced inhibitory action of hesperetin contrasts with lack of alpha-glucosidase inhibition reported earlier for hesperidin, which is the naturally occurring glucoside of hesperetin. However, in the intestine, which is the site of action of alpha-glucosidase, hesperetin is liberated from the sugar residue by bacterial enzymes prior to absorption. Naringenin is cleaved in the gut from its glucoside form by the same mechanism whereas coumarins and polymethoxylated flavones have no sugar residues. As will be understood and as discussed above, herpertin and narnigenin are not PMF compounds.

The above data suggest that tangeretin and other PMFs as well as coumarins found in citrus may exert their beneficial effects in insulin resistance and in Type 2 diabetes at least partly by inhibiting activity of alpha-glucosidase. This effect is postulated and should not be construed as limiting the invention in any way.

b) In Vivo Studies

A second animal study was conducted to determine whether in hamsters with fructose-induced insulin resistance (IR), replacing dietary tangeretin (1% in the diet) with equivalent level of mixed citrus PMF could result in reduction of metabolic abnormalities comparable to that observed with tangeretin. The PMF mixture that was used was as follows:

a) sinensetin—9.3%

b) nobilten—35%

c) tangeretin—11.1%

d) heptamethoxyflavone—33.5%

e) tetramethylscutellarein—11.1%

The additional objective was to evaluate the effect of dietary PMF on glucose tolerance and on serum concentrations of leptin. Hamsters (9-10 per group) were given semipurified, 60% fructose diet with or without 1% PMF, and the control group was fed a standard semipurified diet, which did not produce insulin resistance. After 17-18 days, a glucose tolerance test was performed in fasted animals injected i.p. with 1 g/kg of glucose (6-7 hamsters/group). Serum glucose concentrations were measured before the i.p. injection and in 30 min intervals for 2 h after the injection by using a blood glucose meter. At the end of the feeding study (3 weeks) blood samples were collected by heart puncture for measurement of plasma lipids, glucose, NEFA (non-esterified fatty acids), insulin, nitrites/nitrates (end products of nitric oxide metabolism) and leptin. Total cholesterol in whole serum and in HDL fraction as well as total triacylglycerols and glucose were measured by enzymatic timed-endpoint methods, using the Beckman Coulter reagents and SYNCHRON™ LX System. VLDL+LDL cholesterol concentrations were calculated as a difference between total and HDL cholesterol. NEFA were determined enzymatically by NEFA C kit (Wako Chemicals USA Inc., Richmond, Va.). Serum insulin and serum nitrates/nitrites concentrations were determined using Insulin kit and Nitrate/Nitrite Colorimetric Assay kit from Cayman Chemical Co., Ann Arbor, Mich. Leptin was evaluated with the kit from Assay Designs Inc., Ann Arbor, Mich.

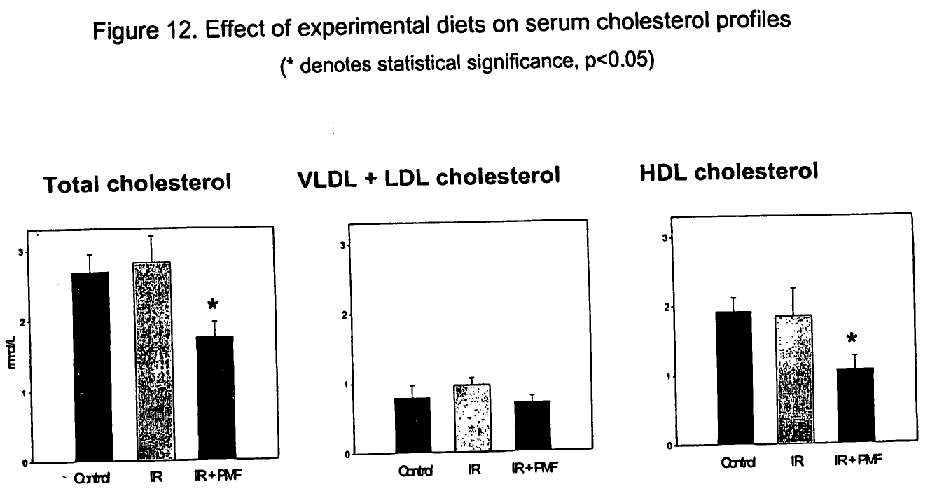

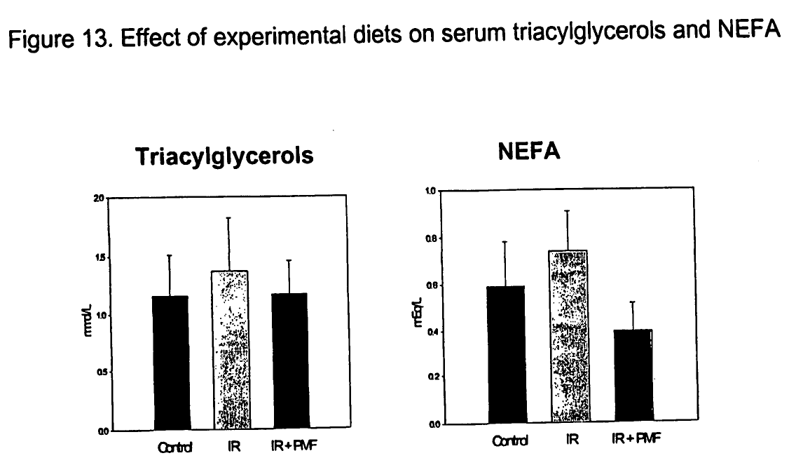

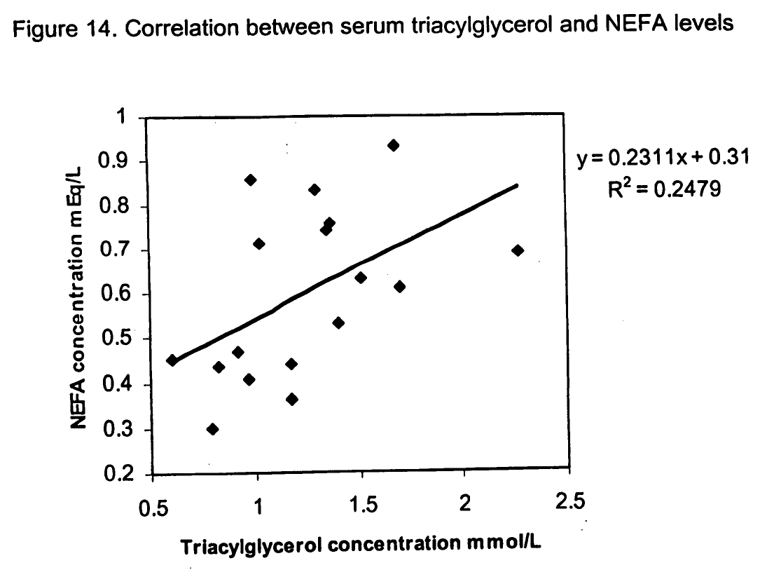

Growth performance data showed no significant difference in growth rate and food consumption between the groups. Replacing the control diet with 60% fructose (IR diet) resulted in moderate increases in serum total and VLDL+LDL cholesterol, triacylglycerols and NEFA (by 5%, 19%, 15% and 20%, respectively). The addition of PMF to the IR diet significantly reduced total, VLDL+LDL and HDL cholesterol and serum NEFA concentrations (by 38%, 28%, 42% and 47%, respectively) and also appeared to reverse fructose-induced increases in serum triacylglycerols as illustrated in Table 4 and FIGS. 12 and 13. The observed changes in serum lipids were generally similar to those demonstrated earlier for tangeretin, but the PMF mixture appeared to have greater beneficial impact on lipoprotein cholesterol. Also, in the present example, as in the previous one, changes in serum triacylglycerol levels were positively correlated with serum NEFA concentrations (r²=0.2479) as illustrated in FIG. 14.

Other metabolic changes associated with feeding experimental diets are summarized in Table 5. Feeding an IR diet marginally increased serum glucose and insulin (by 10% and 7%, respectively) and also increased serum nitrate/nitrite levels by 51%. Addition of the PMF mixture reversed small changes in serum glucose induced by the IR diet and also caused a 26% decrease in serum insulin and a substantial, 175% increase in serum nitrates/nitrites concentration. These changes were similar to those observed earlier in hamster experiment with tangeretin.

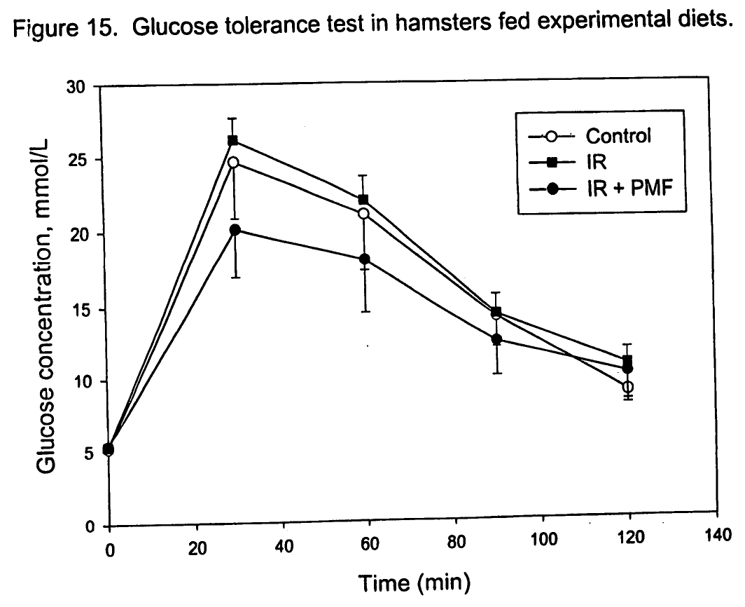

Results of the glucose tolerance test are depicted in FIG. 15 and in Table 6. Glucose levels during the test tended to be reduced in PMF-fed animals, resulting in 21% lower area under the curve and 28% lower maximum serum glucose concentration. This suggests a reduced tendency to develop glucose intolerance (associated with insulin resistance) in hamsters fed PMF-supplemented diet.

### Summary of Results

As indicated above, in fructose-fed hamsters, supplementation with the PMF mixture normalizes metabolic changes associated with insulin resistance. The ability of the PMF mixture to normalize cholesterol levels appears to be better than that observed when tangeretin is used alone.

In the IR hamster model, PMF supplementation also appears to have a beneficial effect on glucose metabolism, reducing glucose intolerance.

The mechanism of action of PMF in insulin resistance may involve inhibition of alpha-glucosidase in the gut. However, this conclusion is postulated and should not be construed as in any way limiting the scope of the present invention.

### References

The following references have been mentioned in the above description. The contents of the following references are incorporated herein by reference.

1. Taghibiglou, C., Carpentier, A., Van Iderstine, S. C., Chen, B., Rudy, D., Aiton, A., Lewis, G. F. and Adeli, K. Mechanism Of Hepatic Very Low Density Lipoprotein Overproduction In Insulin Resistance. J. Biol. Chem. 275 (2000), 8416-8425.

2. Li, H. and Forstermann, U. Nitric Oxide In The Pathogenesis Of Vascular Disease. J. Pathol. 190 (2000), 244-254.

3. Rottiers, R. Diabetes And Nutrition. Inform 11 (2000), 873-877.

4. Kurowska, E. M., Manthey, J. A. and Hasegawa, S. Regulatory Effects Of Tangeretin, A Flavonoid From Tangerines, And Limonin, A Limonoid From Citrus, On Apo B Metabolism In Hepg2 Cells. FASEB J. 14 (2000) A298.

5. Kurowska, E. M., Guthrie, N. and Manthey, J. A. Hypolipidemic Activities Of Tangeretin, A Flavonoid From Tangerines, In Vitro And In Vivo. FASEB J. 15 (2001) A395.

6. Dashiti, N., Williams, D. L. and Alaupovic, P. Effects Of Oleate And Insulin On The Production Rates And Cellular Mrna Concentrations Of Apolipoproteins In Hepg2 Cells. J. Lipid Res. 30 (1989) 1365-1373.

7. Kasim-Karakas, S. E., Vriend, H., Almario, R., Chow, L-C. and Goodman, M. N. Effects Of Dietary Carbohydrates On Glucose And Lipid Metabolism In Golden Syrian Hamsters. J. Lab. Clin. Med. 128 (1996), 208-213.

8. Borradaile, N. M., Carroll, K. K. and Kurowska, E. M. Regulation Of Hepg2 Cell Apolipoprotein B Metabolism By The Citrus Flavanones Hesperetin And Naringenin. Lipids 34 (1999) 591-598.

9. Kurioka, S., Koshimura, K., Murakami, Y., Nishiki, M. and Kato, Y. Reverse Correlation Between Urine Nitric Oxide Metabolites And Insulin Resistance In Patients With Type 2 Diabetes Mellitus. Endocr. J. 47 (2000), 77-81.

10. Oshida, Y., Tachi, Y., Morishita, Y., Kitakoshi, K., Fuku, N., Han, Y. Q., Oshawa, I. and Sato, Y. Nitric Oxide Decreases Insulin Resistance Induced By High-Fructose Feeding. Horm. Metab. Res. 32 (2000), 339-342.

11. Kim, J-S, Kwon, C-S, Son, K. H. Biosci. Biotechnol. Biochem. 64, 2000, 2458-2461.

Although the invention has been described with reference to certain specific embodiments, various modifications thereof will be apparent to those skilled in the art without departing from the spirit and scope of the invention as outlined in the claims appended hereto.

TABLE 1
Growth Performance Of Hamsters Fed Experimental Diets

| | Initial Weight | Growth Rate | Food Consumption |
|---|---|---|---|
| Diet | (g) | (g/day) | (g/day) |
| Control | 134.0 ± 8.0 | 0.60 ± 0.30 | 6.93 ± 0.76 |
| Fructose | 134.0 ± 8.9 | 0.43 ± 0.36 | 6.12 ± 0.76 |
| +0.25% PMF | 133.8 ± 7.5 | 0.50 ± 0.21 | 6.59 ± 0.47 |
| +0.50% PMF | 133.9 ± 9.0 | 0.56 ± 0.40 | 6.42 ± 1.07 |
| +1.00% PMF | 133.9 ± 8.8 | 0.19 ± 0.27 | 6.48 ± 1.05 |

Values are means ± SD.

TABLE 2
Metabolic Changes Associated With Feeding Experimental Diets

| | Total | VLDL + LDL | HDL | | | | |
|---|---|---|---|---|---|---|---|
| | cholesterol | cholesterol | cholesterol | Triacylglycerols | NEFA | Insulin | NO₂/NO₃ |
| Diet (n) | mmol/L | mmol/L | mmol/L | mmol/L | mEq/L | pmol/L | μmol/L |
| Control (9) | 2.65 ± 0.59 | 0.87 ± 0.21 | 1.78 ± 0.50 | 1.19 ± 0.49 | 1.43 ± 0.28 | 651 ± 338 | 50.4 ± 13.8 |
| % change | −26% | | −44% | −67% | −35% | −23% | |
| Fructose (8) | 3.35 ± 0.53 | 0.79 ± 0.36 | 2.57 ± 0.55 | 1.99 ± 0.64 | 1.93 ± 0.54 | 842 ± 197 | 53.4 ± 13.3 |
| +0.25% Tan (10) | 3.17 ± 0.69 | 0.62 ± 0.3 | 2.55 ± 0.72 | 1.51 ± 0.56 | 1.48 ± 0.36 | 735 ± 315 | 55.2 ± 18.8 |
| % change | −5% | | 0% | −24% | −23% | −13% | |
| +0.5% Tan (8) | 2.61 ± 0.43* | 0.68 ± 0.28 | 1.93 ± 0.48 | 1.45 ± 0.52 | 1.50 ± 0.33 | 729 ± 239 | 52.0 ± 18.5 |
| % change | −22% | | −17% | −27% | −22% | −13% | |
| +1.0% Tan (10) | 2.69 ± 0.23* | 0.81 ± 0.22 | 1.88 ± 0.26* | 1.35 ± 0.57 | 1.58 ± 0.32 | 675 ± 296 | 106.3 ± 27.9* |
| % change | −20% | | −27% | −32% | −18% | −20% | +99% |

Values are means ± SD.
*significantly different from fructose group by ANOVA, p < 0.05.

TABLE 3
IC₅₀ Values For In Vitro Inhibition Of Alpha-Glucosidase
By Citrus Flavanones, Coumarins And PMF.

| Compound | IC₅₀ μg/mL | IC₅₀ mM/L |
|---|---|---|
| Hesperetin | 12.1 | 0.04 |
| Coumarins* | 83.5 | 0.28 |
| Naringenin | 84.4 | 0.31 |
| Heptamethoxyflavone | 201.7 | 0.50 |
| Tangeretin | 230.0 | 0.67 |
| Nobiletin | 530.2 | 1.42 |

*contain mostly auraptene.

TABLE 4
Changes In Blood Lipids Associated With Feeding Experimental Diets

| | Total cholesterol | VLDL + LDL | HDL cholesterol | Triacylglycerols | NEFA |
|---|---|---|---|---|---|
| Diet (n) | mmol/L | cholesterol, mmol/L | mmol/L | mmol/L | mEq/L |
| Control (10) | 2.69 ± 0.25 | 0.78 ± 0.19 | 1.92 ± 0.19 | 1.16 ± 0.35 | 0.59 ± 0.19 |
| % change | −5% | −19% | +3% | −15% | −20% |
| Fructose (9) | 2.82 ± 0.38 | 0.96 ± 0.11 | 1.86 ± 0.38 | 1.37 ± 0.45 | 0.74 ± 0.17 |
| Fructose + PMF (9) | 1.76 ± 0.21 | 0.69 ± 0.10 | 1.07 ± 0.21 | 1.17 ± 0.29 | 0.40 ± 0.12 |
| % change | −38% | −28% | −42% | −15% | −47% |

TABLE 5
Other Metabolic Changes Associated With Feeding Experimental Diets

| | Serum glucose | | Nitrates/nitrites | |
|---|---|---|---|---|
| Diet (n) | mmol/L | Serum insulin ng/mL | mmol/L | Leptin |
| Control (10) | 11.66 ± 4.35 | 0.209 ± 0.113 | 3.93 ± 1.81 | |
| % change | −10% | −7% | −51% | |
| Fructose (9) | 12.91 ± 3.93 | 0.224 ± 0.113 | 8.00 ± 3.35 | |
| Fructose + PMF (9) | 11.20 ± 2.70 | 0.165 ± 0.043 | 21.97 ± 0.40 | |
| % change | −13% | −26% | +175% | |

TABLE 6
Diet-Induced Changes In Pharmacokinetics Of Serum Glucose
In Hamsters Injected I.P. With Glucose And Followed For 2 H.

| | Control | IR control | IR + PMF |
|---|---|---|---|
| AUC₀₋₂ₕ | 2136.3 ± 726.3 | 2255.8 ± 356.6 | 1790.8 ± 860.5 |
| mmol/L × | | | |
| min* | | | |
| % difference | −5% | | −21% |
| from | | | |
| IR group | | | |
| Cₘₐₓ | 25.6 ± 10.0 | 26.6 ± 4.0 | 19.2 ± 9.0 |
| mmol/L** | | | |
| % difference | −4% | | −28% |
| from | | | |
| IR group | | | |

*AUC₀₋₂ₕ - area under the curve from 0 to 120 min.
**Cₘₐₓ - maximum serum concentration.

The embodiments of the invention in which an exclusive property or privilege is claimed are defined as follows:

1. A pharmaceutical composition for treating a mammal having metabolic abnormalities resulting from insulin resistance comprising an effective amount of at least one polymethoxyflavone compound and a suitable pharmaceutically acceptable diluent, carrier or adjuvant.

2. The composition of claim 1 wherein said polymethoxyflavone is chosen from sinensetin, nobilten, tangeretin, heptamethoxyflavone, tetramethylscutellarein and mixtures thereof.

3. The composition of claim 2 wherein said polymethoxyflavone is tangeretin.

4. The composition of claim 1 wherein said at least one polymethoxyflavone comprises a mixture of various polymethoxyflavone compounds.

5. The composition of claim 4 wherein said mixture comprises sinensetin, nobilten, tangeretin, heptamethoxyflavone, and tetramethylscutellarein.

6. The composition of claim 1 wherein said composition is prepared for administration by a means chosen from oral, transdermal, rectal, intravenous, intramuscular, intraperitoneal subcutaneous, topical, or by inhalation.

7. The composition of claim 1 wherein said composition is administered orally.

8. The use of a metabolic abnormality reducing amount of at least one polymethoxyflavone in a mammal experiencing insulin resistance syndrome.

9. The use as claimed in claim 8 wherein said polymethoxyflavone is chosen from sinensetin, nobilten, tangeretin, heptamethoxyflavone, tetramethylscutellarein and mixtures thereof.

10. The use as claimed in claim 8 wherein said polymethoxyflavone is tangeretin.

11. The use as claimed in claim 8 wherein said at least one polymethoxyflavone comprises a mixture of various polymethoxyflavone compounds.

12. The use as claimed in claim 11 wherein said mixture comprises sinensetin, nobilten, tangeretin, heptamethoxyflavone, and tetramethylscutellarein.

13. The use as claimed in claim 8 wherein said at least one polymethoxyflavone is administered by a means chosen from oral, transdermal, rectal, intravenous, intramuscular, intraperitoneal subcutaneous, topical, or by inhalation.

14. The use as claimed in claim 8 wherein said at least one polymethoxyflavone is administered orally.

15. A method of treating a mammal having metabolic abnormalities resulting from insulin resistance comprising administering an effective amount of at least one polymethoxyflavone compound.

16. The method of claim 15 wherein said polymethoxyflavone is chosen from sinensetin, nobilten, tangeretin, heptamethoxyflavone, tetramethylscutellarein and mixtures thereof.

17. The method of 15 wherein said polymethoxyflavone is tangeretin.

18. The method of claim 15 wherein said at least one polymethoxyflavone comprises a mixture of various polymethoxyflavone compounds.

19. The method of claim 18 wherein said mixture comprises sinensetin, nobilten, tangeretin, heptamethoxyflavone, and tetramethylscutellarein.

20. The method of claim 15 wherein said at least one polymethoxyflavone is administered by a means chosen from oral, transdermal, rectal, intravenous, intramuscular, intraperitoneal subcutaneous, topical, or by inhalation.

21. The method of claim 15 wherein said at least one polymethoxyflavone is administered orally.

22. The method of claim 15 wherein said at least one polymethoxyflavone is administered to said mammal in an amount of up to 5000 mg/day.

23. The method of claim 22 wherein said at least one polymethoxyflavone is administered to said mammal in an amount of up to 70 mg/kg/day, based on the weight of said mammal.
